(19) United States
(12) Patent Application Publication
(10) Pub. No.: US 2011/0237444 A1
(43) Pub. Date: Sep. 29, 2011

(54) METHODS OF MAPPING GENOMIC METHYLATION PATTERNS

(75) Inventors: Kevin Clancy, Oceanside, CA (US); Gavin Meredith, Cardiff, CA (US); Christopher Adams, Solana Beach, CA (US); Daniel Krissinger, San Diego, CA (US)

(73) Assignee: LIFE TECHNOLOGIES CORPORATION, Carlsbad, CA (US)

(21) Appl. No.: 12/950,227

(22) Filed: Nov. 19, 2010

Related U.S. Application Data
(60) Provisional application No. 61/263,194, filed on Nov. 20, 2009.
(60) Provisional application No. 61/411,866, filed on Nov. 9, 2010.

(51) Int. Cl. C40B 30/00; C12Q 1/68; C07K 17/00

(52) U.S. Cl. 506/7; 435/6.11; 530/391.1

(57) ABSTRACT

The invention relates to sample analysis work flows for increasing the efficiency of experiments. Compositions and methods are described for selectively increase the abundance of methylated nucleic acid over non-methylated nucleic acid, followed by analysis of the nucleic acid to identify methylation sites.

### CROSS-REFERENCE TO RELATED APPLICATIONS

This application claims the benefit of U.S. Provisional Application Nos. 61/623,194, filed Nov. 20, 2009 and 61/411, 866 filed Nov. 9, 2010, the disclosures of which are incorporated herein by reference in their entireties.

### BACKGROUND

The classic method for single-base resolution of the cytidine methylation that occurs in mammalian DNA involves the use of sodium bisulfite to chemically convert non-methylated cytidines to uridines. After conversion, the DNA is amplified, typically by PCR, and in this process the uridines are re-encoded as thymidines. The DNA is then Sanger-type sequenced either directly or sequenced from bacterial clones that have been transformed with a cloning vector that contains a single-copy of the original DNA. Sequences derived from this workflow are compared to reference (non-converted) sequence and C to T “mutations” are interpreted as representing cytidines that were non-methylated in the original sample; conversely, cytidines that persist through this workflow are interpreted as having been methylated in the original sample. This workflow, commonly referred to as “bisulfite sequencing” is widely regarded within the field as the “gold-standard” for DNA methylation analysis.

Recently, high throughput sequencing as enabled by “next generation” platforms such as Life Technologies' SOLiD™ system, Illumina's Genome Analyzer, and Roche's 454 system has been coupled with sodium bisulfite conversion to provide single-base resolution of the positions of cytidine methylation on whole or partial genomes (see Lister and Ecker, Genome Res., 19:959-966 (2009) and references therein). This analysis is hindered for three major reasons. First, it is currently relatively expensive to sequence a human genome at a sufficient depth of coverage to determine all or most of the cytidine methylation from a given sample since this requires approximately 90 Gigabases of sequencing for 30-fold coverage of the ˜3 Gigabase human genome. Second, human genomes are variable at multiple levels. Not only does this include the exact methylation pattern for a given sample but it also includes a high incidence of copy-number variation (CNV) and the occurrence of insertions and deletions (indels) and inversions, repeats, translocations and single-nucleotide polymorphisms (SNPs) and complex combinations of these changes and rearrangements. Again, to properly understand the context of DNA methylation within a sample some degree of de novo sequencing of the sample may be required. Finally, because the bisulfite conversion reaction typically changes 99% of the cytidines to uridines which are then converted to thymidines by DNA amplification, the “complexity” of the sequence information becomes significantly reduced. This makes subsequent alignment and mapping of the sequencing data computationally more difficult and further prompts the need for even more sequencing and hence more expense.

### SUMMARY

Described herein is a modified workflow for the analysis of nucleic acid methylation in the genome of an organism. Sequencing of a portion of the genome which is enriched in methylated DNA provides a reduced representation of the whole genome that may be “focused” on the sequences that harbor methylation. Such a subset of sequences, relative to the whole genome, may be referred to as the “methylation territory”. A methylation territory that is sequenced in this manner may also capture evidence of variability within a sample genome as it relates to the methylation pattern, for example translocation junctions if they happen to occur near methylated CpGs. Sequencing of methylation enriched sequences may yield sequences that carry a reduced load of C to T converted bases because the sequences carry significant amounts of methylated cytidine which are not converted. This may aid in mapping of sequencing reads in regions having reduced complexity as a result of extensive conversion of C to T. Also, mapping within the methylation territory may reduce the amount of computation required and the uncertainty of alignment compared to mapping un-enriched fragments.

In some embodiments, the invention includes methods of mapping methylated bases (e.g., cytidines) in the genome of an organism. In some specific embodiments, such methods involve one or more of the following steps, (a) isolating methylated nucleic acid (e.g., methylated DNA) fragments from the organism, (b) sequencing a first portion of the methylated nucleic acid fragments isolated from the genome of the organism thereby producing a first nucleic acid sequence, (c) sequencing a second portion of the methylated nucleic acid isolated from the genome of the organism which has been treated such that non-methylated cytidine is converted to uridine or thymidine thereby producing a second nucleic acid sequence, and/or (d) aligning the second nucleic acid sequence with the first nucleic acid sequence thereby producing a map of methylated and non-methylated cytidine in the genome of the organism.

In other embodiments, such methods involve one or more of the following steps (a) isolating from the genome of the organism methylated nucleic acid fragments, (b) splitting the isolated methylated nucleic acid fragments into at least a first portion and a second portion, (c) treating the first portion of isolated methylated nucleic acid fragments such that non-methylated cytidine is converted to uridine or thymidine, (d) sequencing the first and second portions of isolated methylated nucleic acid, and/or (e) mapping the sequence of the first portion of the isolated methylated nucleic acid to the sequence of the second portion of the isolated methylated nucleic acid.

In particular embodiments, nucleic acid may be either DNA or RNA. In further embodiments the nucleic acid sample may be fragmented. Such nucleic acid fragments may be up to 50 bp, 100 bp, 200 bp, 300 bp, 400 bp, 500 bp, 600 bp, 700 bp, 800 bp, 900 bp or 1000 bp in length (e.g., average length in the population of nucleic acid fragments).

In some embodiments, methylated nucleic acid fragments may be isolated using methyl binding proteins (MBPs). In certain embodiments, the methylated nucleic acid fragments may be isolated using antibodies specific for methylated nucleic acid. Thus, in further embodiments, the methyl binding protein (e.g., methylated nucleic acid specific antibodies) or other methylated nucleic acid specific ligands may be bound directly or indirectly to a solid support. For embodiments using indirect binding, a methylated nucleic acid binding ligand may be labeled with a molecule such as biotin which may be captured by a second molecule such as avidin or streptavidin which may in turn be bound to a solid support. Antibodies specific for a methylated nucleic acid binding protein or antibody specific for methylated nucleic acid may also be used to indirectly bind methylated nucleic acid to a solid support.

Suitable solid supports for binding methylated nucleic acid include, but are not limited to, agarose, sepharose, polyacrylamide, agarose/polyacrylamide co-polymers, dextran, cellulose, polypropylene, polycarbonate, nitrocellulose, glass, silica, paper. A solid support may be in the form of particles, beads, magnetic or paramagnetic beads, slides, multi-well plates, tubes, vials, and pipette tips.

Nucleic acid fragments may be isolated from prokaryotic organisms such as bacteria or from eukaryotic organisms including but not limited to yeast, plants, insects, fish, mammals, rodents, primates, and humans. In some embodiments the nucleic acid fragments may be isolated from specific organs, tissues or cells and in further embodiments these organs, tissues or cells may be from organisms at different stages of development including stages of embryonic development. In other embodiments the organs, tissues or cells may be healthy or diseased such as from a tumor. The organs, tissues or cells may also have been exposed to hormones, cytokines, chemokines or other natural or synthetic chemical compounds.

In some embodiments, the nucleic acid may be methylated at one or more cytidines or adenosines. In other embodiments, the nucleic acid may be hydroxymethylated on one or more cytidines. In other embodiments, the nucleic acid may be methylated on one or more guanosines, uridines, or thymidines and in some embodiments the nucleic acid may contain one or more of any of these modified bases. In embodiments where methylation or hydroxymethylation is at the 5-carbon position of cytidine, non-methylated or non-hydroxymethylated cytidine may be deaminated while methylated cytidine remains unchanged. In some embodiments bisulfite may be used to deaminate the methylated or hydroxymethylated nucleic acid. In further embodiments, the nucleic acid contains one or more of the various known chemical modifications such as described in the texts Principles of Nucleic Acid Structure by W. Sanger (1984) and Nucleic Acids: Structures, Properties, and Functions by V. A. Bloomfield, D. M. Crothers, and I. Tinoco, Jr. (2000).

In some embodiments of the invention the isolated methylated (or hydroxymethylated) nucleic acid fragments may be amplified prior to sequencing, for example by the use of polymerase chain reaction or other amplification methods. In order to preserve the distribution of methylated cytidines within the nucleic acid, amplification may occur after conversion of non-methylated cytidines to uridines with bisulfite.

Sequencing of the methylated nucleic acid fragments, either before or after treatment to convert non-methylated bases may be performed by any of the standard methods known in the art. Suitable methods include chain termination methods (Sanger sequencing), Maxim-Gilbert sequencing, and high throughput methods such as the SOLiD system (Life Technologies, Carlsbad, Calif.); Genome Sequencer FLX system, commonly known as 454-sequencing (Roche Diagnostics, Indianapolis, Ind.); the Solexa/Illumina Genome Analyzer (Illumina, San Diego, Calif.); and the Helicos Genetic Analysis System (Helicos Biosciences, Cambridge, Mass.).

Additional embodiments may comprise a kit for mapping methylated cytidine in a genome of an organism comprising a methylated DNA binding substance bound to a solid support. A kit may further comprise any one or a combination of the following; one or more buffers for binding the methylated DNA to the DNA binding substance, one or more buffers for eluting the bound methylated DNA from the methylated DNA binding substance, reagents for converting methylated cytidine to uridine, and a written manual describing data analysis procedures for mapping methylated cytidine in a genome of an organism.

### BRIEF DESCRIPTION OF THE DRAWINGS

FIG. 1 shows a diagram comparing conventional determination of methylation patterns to a method using a methylation territory map, in accordance with some embodiments.

FIG. 2A depicts a flow diagram for the analysis of sequencing reads of a reference sequence and a bisulfite converted sequence using a methylation territory mapping approach, in accordance with some embodiments.

FIG. 2B depicts a flow diagram for post-mapping analysis of METHYLMINER™ enriched and bisulfate converted reads, in accordance with some embodiments.

FIG. 3 depicts a METHYLMINER™ enriched methylation territory map and the use of this territory to align bisulfite converted SOLiD sequencing reads, in accordance with some embodiments. (Part A) Illustration of a methylation territory derived from 500 mM MethyMiner™ eluted DNA sample (red bars) compared to a complete genomic reference sequence (green bar) and an illustration of bisulfite converted reads aligning to the territory (black bars). (Part B) Bisulfite-converted reads mapping within 500 mM and 1000 mM enriched fractions (i.e., methylated territories) respectively. Shown is a diagram of 500 mM (red bars) and 1000 mM (black bars) METHYLMINER™ enriched methylated territories within a defined region of chromosome 21 and the bisulfite converted sequencing reads that map within each of these territories. Also shown are the areas where the 500 mM and 1000 mM territories overlap (black bars) and the bisulfite sequencing reads that map within this region. Green bars represent annotated CpG islands.

FIG. 4 depicts representative experimentally determined aligned SOLiD sequencing reads of a bisulfite converted sample compared to the unconverted reference sequence and a computationally determined bisulfite converted reference sequence from a region of methylation territory from chromosome 21, in accordance with some embodiments.

### DETAILED DESCRIPTION

The methods disclosed herein provide, in part, for the isolation of nucleic acid from organisms, enrichment of the isolated nucleic acid based on chemical modification of the nucleic acid, fragmentation of the nucleic acid, modifying or otherwise interacting with the chemical modification present on the nucleic acid and sequencing the nucleic acid so that the pattern of the chemical modification within the nucleic acid may be identified.

As used herein, the term “methylated”, when used in reference to nucleic acid, refers to nucleic acid which contains a methyl group on a base which is not normally present in nucleic acid when it is generated. In most cases, this base will be a cytidine and the methylated form will be 5-methylcytidine (“5-mCyt”). In some case, adenosine may be methylated. The term methylated includes hemi-methylated and fully methylated nucleic acid.

As used herein, the term “nucleic acid” refers to a sequence of contiguous nucleotides (riboNTPs, dNTPs, ddNTPs, or combinations thereof) of any length (e.g., complete chromosomes and/or genomes). A nucleic acid molecule may encode a full-length polypeptide or a fragment of any length thereof, or may be non-coding (e.g., may be a promoter or enhancer).

As used herein, the term “genome” refers to the entire genetic complement of an organism. In the case of eukaryotic organisms, genome refers to the nucleic acid molecules found in both the nucleus of the cell and in the mitochondria. A genome includes both coding and non-coding nucleic acid sequences. Genomes, when appropriate, are composed of both chromosomal and non-chromosomal nucleic acids.

As used herein, the term “methyl binding protein”, or an “MBP”, is a protein or peptide that specifically binds to a nucleic acid with one or more methylated base residues, such as a protein or peptide that binds to methylated CpG islet(s) in a nucleic acid (e.g., preferentially binds to a nucleotide sequence which containing one or more methylated CpG dinucleotides over the same nucleotide sequence which is not methylated). Examples of MBP include, but are not limited to, the methylated-CpG binding protein 2 (MeCP2) and the methyl-CpG-binding domain proteins MBD1, MBD2, MBD3, and MBD4, and their homologs (with at least 80% sequence identity, at least 90% sequence identity, or at least 95% sequence identity, e.g., to human, mouse, or rat MeCP2, MBD1, MBD2, MBD3, MBD4. or Kaiso) that bind to methylated DNA. Exemplary MBPs include, e.g., the methylated DNA binding domains from such proteins (e.g., from MeCP2, MBD1, MBD2, MBD3, or MBD4) and other truncated and/or mutant versions of the proteins as well as the full length wild-type proteins (see Ballestar and Wolffe, Eur. J. Biochem. 268:1-6 (2001); Chen et al., Science 302:885-889 (2003) and supplemental materials S1-S13; Jorgensen et al., Nucl. Acids. Res. 34:e96 (2006); and Valls et al., Cancer Res. 68:7258-7263 (2008). Exemplary MBPs also include antibodies that bind specifically to methylated nucleic acid (see, e.g., Sano et al., Proc. Natl. Acad. Sci. USA 77:3581-3585 (1980) and Storl et al., Biochem. Biophys. Acta 564:23-30 (1979)), or the MBP can be a polypeptide other than an antibody. Additional MBP sequences can be found, for example, in Genbank and in the literature.

As used herein, the term “methylation specific enrichment” refers to processes which result in the increase in ratio of methylated nucleic acid over non-methylated nucleic acid. Typically, such enrichment will be in ranges from about 5 fold to about 200 fold, from about 5 fold to about 40 fold, from about 5 fold to about 30 fold, from about 5 fold to about 20 fold, from about 5 fold to about 15 fold, from about 5 fold to about 10 fold, from about 10 fold to about 200 fold, from about 10 fold to about 100 fold, from about 10 fold to about 60 fold, from about 10 fold to about 50 fold, from about 10 fold to about 30 fold, etc.

As used herein, the term “hypermethylation” refers to the average methylation state corresponding to an increased presence of methylated bases (e.g., 5-mCyt) at one or a plurality of locations (e.g., CpG dinucleotides) within a nucleotide sequence, relative to the amount of methylated bases (e.g., 5-mCyt) found at corresponding location within a normal control nucleic acid sample. “Hypomethylation” is similar but relates to a decreased (vs. increased) presence of methylated bases.

As used herein, the term “methylation assay” refers to any assay for determining the methylation state of one or more nucleotide sequences (e.g., CpG dinucleotide) sequences within a nucleic acid molecule. One example of a methylation assay is bisulfite sequencing.

In some embodiments, the invention includes work flows for the processing of nucleic acid samples. Exemplary work flows may involve one or more of the following steps: (a) the generation of one or more (e.g., one, two, three, four, five, eight, ten, etc.) samples containing nucleic acid, (b) fragmentation of nucleic acid in the one or more samples, (c) enrichment of nucleic acid of interest (e.g., methylated nucleic acid) in the one or more samples, (d) separation of each sample into two or more (e.g., two, three, four, five, eight, ten, etc.) portions, (e) treatment (e.g., bisulfite treatment) of one portion of each sample but not the other portion, (f) analysis (e.g., similar or identical analysis) of at least two of the two or more portions of each sample, and/or (g) comparison of data (e.g., sequence data) derived from at least two of the two or more portions of each sample. In many embodiments of the invention, treatment and/or analysis referred to above will be related to the detection of methylated bases.

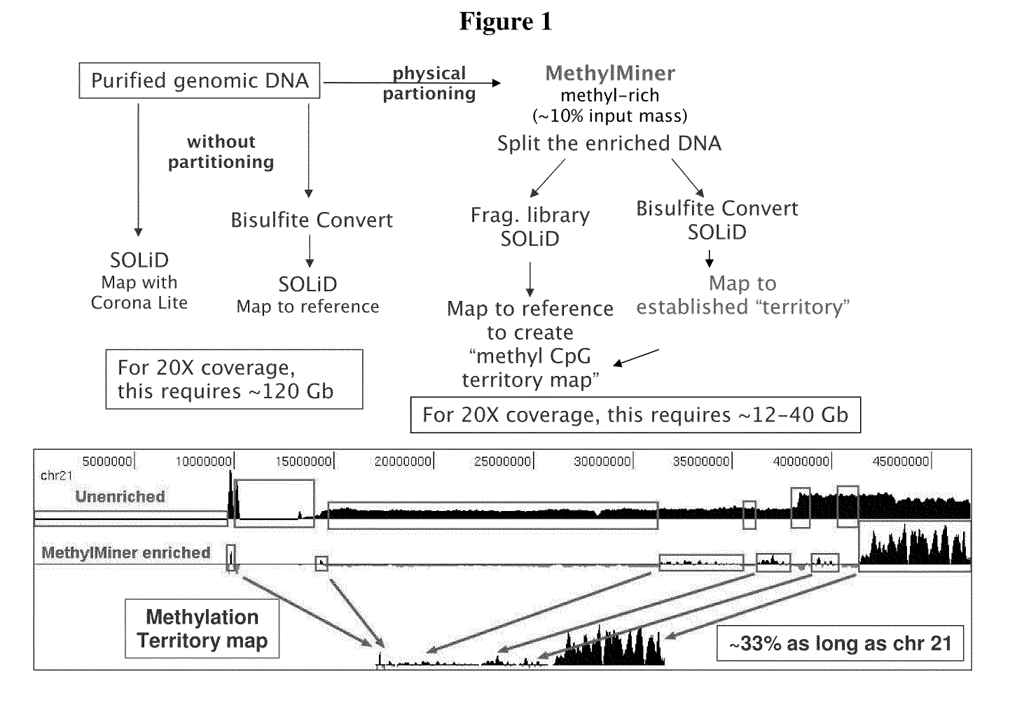

FIG. 1 depicts a comparison of a conventional analysis of a methylation profile for human chromosome 21 to analysis of a methylation profile using enrichment for methylated DNA and the use of a methylation territory map. For conventional methylation analysis depicted on the upper left-hand portion of FIG. 1, sequencing data is obtained from both native and bisulfite converted genomic DNA. In order to achieve 20× coverage for sequencing of human chromosome 21, approximately 120 gigabases would need to be sequenced. One embodiment of methods described herein is depicted in the upper right-hand corner of FIG. 1. In this embodiment, a sample of methylation enriched DNA may be split into two portions. One portion may be sequenced and mapped to a reference sequence to create a methylation territory map. Such a map is depicted at the bottom of FIG. 1. The remaining portion of methylation enriched DNA may be bisulfite converted, sequenced, and the sequence mapped to a methylation territory. Using this approach, 20× coverage of a methylation territory of human chromosome 21 would require sequencing approximately 12-40 gigabases, at least a three fold reduction compared to the conventional approach.

The invention thus provides methods for increasing the efficiency of nucleic acid analysis. This efficiency may be achieved by decreasing the amount of nucleic acid which needs to be screened to obtain desired data. For example, using the schematic in FIG. 1 for purposes of illustration, experiments which result in the generation of 120 gigabytes of data can be designed to yield only 40 gigabytes of data while achieving the same or substantially similar goal (e.g., the identification of methylation sites in a genomic DNA sample). The net result here is a 66% decrease in the amount of data generated, along with a corresponding reduction in reagent usage and bench time. In a particular embodiment, the invention is directed to work flows which result in at least a 50%, 60%, 70%, 80%, 85%, etc. (e.g., from about 50% to about 95%, from about 60% to about 95%, from about 70% to about 95%, from about 80% to about 95%, from about 50% to about 85%, from about 50% to about 75%, from about 60% to about 90%, from about 60% to about 85%, etc.) decrease in the amount of data generated. One method by which such reductions in generated data may be achieved is by focusing analysis on nucleic acid molecules which have been enriched for a particular feature (e.g., the feature of interest, such as the presence of methyl groups). In addition to reductions in the amount of data generated, the invention also provides similar reductions in reagent use and bench time. Bench time includes equipment use time (e.g., the time need to analyze a sample on a genome sequencer).

The nucleic acid used in the practice of the invention may be DNA or RNA or both. The nucleic acid may be from a variety of organisms including, but not limited to, bacteria, eukaryotes, yeast, plants, insects, vertebrates, rodents, primates, and humans. In the case of higher eukaryotes, nucleic acid may be isolated from individual organs or tissues such as blood, lymph nodes, spleen, lung, skin, liver, kidney, brain, and bone marrow. Nucleic acid may also be isolated from cultured tissues or cells. Nucleic acid may also be isolated from archived medical samples, archived biological samples, environmental samples, or forensic samples. In some embodiments, tissues or cells used as the source of nucleic acid may be from different stages of development or from diseased tissue such as a tumor.

Any of a variety of methods for the isolation of nucleic acid known in the art may be used for the methods described herein. These include kits such as the PURELINK™ Genomic DNA Kits supplied by Life Technologies Corp. (Carlsbad, Calif.). Isolated DNA may be fragmented prior to analysis. Nucleic acid fragmentation may be by any suitable method known in the art including enzymatic methods such as cleavage by restriction enzymes and mechanical methods such as shearing or sonication. Fragmentation of nucleic acid may be to an average size of less than 1000 bp, less than 900 bp, less than 800 bp, less than 700 bp, less than 600 bp, less than 500 bp, less than 400 bp, less than 300 bp, or less than 200 bp. In some instances, nucleic acid fragments used in the practice of the invention may be from about 50 to about 2,000, from about 100 to about 2,000, from about 150 to about 2,000, from about 200 to about 2,000, from about 400 to about 2,000, from about 800 to about 2,000, from about 50 to about 1,500, from about 50 to about 1,000, from about 50 to about 600, from about 50 to about 500, from about 50 to about 300, from about 50 to about 250, from about 100 to about 1,000, from about 100 to about 800, from about 100 to about 500, from about 100 to about 350, from about 100 to about 250, from about 150 to about 500, from about 150 to about 350, etc. bps in length. Further, in some instances, the average size of nucleic acid fragments will fall within such ranges. Also, in some instances, the majority (e.g., greater than 50%, greater than 60%, greater than 70%, greater than 80%, greater than 90%, greater than 95%, greater than 98% etc.) of nucleic acid fragments present will fall within such ranges.

A variety of methods available for the enrichment of methylated DNA, including the use of methylation binding proteins. In humans and other placental mammals, methylation of cytidines at the 5 carbon on the cytidine ring is most commonly found in the sequence context of CG dinucleotides (CpGs), so enrichment that utilizes a methylation CpG binding protein (e.g., methylated CpG binding protein or specific antibody). This enrichment may allow for more of the sequencing reads to be focused on the sequences of interest with a proportionate reduction in the total amount of sequencing that needs to be carried out (and paid for) to achieve sufficient depth of coverage in the regions of interest. Since the number of calculations needed to align experimental sequences scales approximately exponentially with the size of the reference sequence, 10-fold enrichment will thus require ˜ 1/10^{th} to ˜ 1/100^{th} the amount of alignment calculation. With 10-fold enrichment, adequate coverage of the methylation territory can be achieved with ˜ 1/10^{th} to ˜ 1/100^{th} the sequencing time and cost. Because the bisulfite converted DNA needs to be sequenced at about the same level, the total cost and time of sequencing will be reduced to about ⅕^{th} to 1/50^{th} that which is necessary for the established “shotgun” methods of high throughput bisulfite sequencing, particularly with the SOLiD™ platform.

Further, sequencing of the enriched DNA, prior to bisulfite conversion, may provide some measure of the variability that is unique to the sample relative to the established reference human genome sequences (hg18 and hg19); in particular, SNPs, which are common, can be identified. This may be of particular importance in situations where an SNP represents a C to T mutation in the sample relative to the reference. Failure to identify such a SNP can result in inappropriate interpretation of a T in a bisulfite-converted sample as having been a non-methylated C in the unconverted sample. All of these factors may contribute to reduce the time and cost needed to determine a cytidine methylation pattern for any given sample. Further, this approach is not necessarily limited to CpG methylation, but may be broadened to include non-CpG cytidine methylation with appropriate enrichment technologies, such as with the commonly used anti-5-methyl cytosine antibodies that have been described in the literature and offered by commercial vendors.

Kits for isolation of methylated DNA are available commercially, for example the METHYLMINER™ Methylated DNA Enrichment Kit (Life Technologies Corp., Carlsbad, Calif.); METHYLCOLLECTOR™, (Active Motif Inc., Carlsbad, Calif.); Methylated-DNA IP Kit, (Zymo Research, Orange Calif.); METHYLMAGNET™ mCpG DNA Isolation Kit (Ribomed, Carlsbad, Calif.); and METHYLAMP™ Methylated DNA Capture Kit, (Epigentek, Brooklyn, N.Y.).

The METHYLMINER™ kit (Invitrogen catalog no. ME10025) may be used as an illustrative example. The capture medium used in the kit is the methyl-CpG binding domain (MBD) of the human MBD2 protein coupled to superparamagnetic Dynabeads® M-280 Streptavidin via a biotin linker. Typically, this kit can create an enrichment of 4-20 fold by mass, i.e., 75-95% of sample eukaryotic genomic DNA may be isolated as depleted of methylated sequences and 3-20% of sample DNA mass may be isolated as enriched for methylated sequences. A detailed protocol is provided by the manufacturer but briefly, for each μg of isolated and fragmented DNA 10 μl of Dynabeads® M-280 Streptavidin and 3.5 μg of MBD-Biotin protein is used. The reaction conditions may be scaled to use between 5 ng and 25 μg of DNA. After washing the Dynabeads, 3.5 μg of MBD-Biotin protein is added to the beads in a final volume of 200 μl in a 1.7 ml microcentrifuge tube and incubated at room temperature on a rotary mixer for 1 hour.

After incubating the beads with the MBD-Biotin, the beads are washed and the fragmented DNA sample is added at a concentration of 25 ng/μl and final volume of 500 μl of binding buffer. The beads are then incubated at room temperature on a rotary mixer for 1 hour. In order to collect the non-methylated DNA from the sample, the microcentrifuge tube is placed in a magnetic rack for one minute and the supernatant containing the non-methylated DNA is removed and placed in a separate tube for storage.

After further washing, methylated DNA is eluted from the beads by resuspending the beads in 400 μl of 2 M NaCl and incubating on a rotary mixer for 3 minutes. The microcentrifuge tube is then placed in a magnetic rack until all of the beads have accumulated on an inside wall of the tube and the supernatant containing the methylated DNA is collected and transferred to a separate clean microcentrifuge tube. Alternatively, bound methylated DNA may be recovered using proteinase K treatment. In this protocol the beads are resuspended in 200 μl of binding buffer and 0.8 units of Proteinase K is added and the beads are incubated at 57° C. for 90 minutes with agitation. The beads are then placed in a magnetic rack for one minute and the supernatant transferred to a separate tube. This step may be repeated to recover any residual bound DNA.

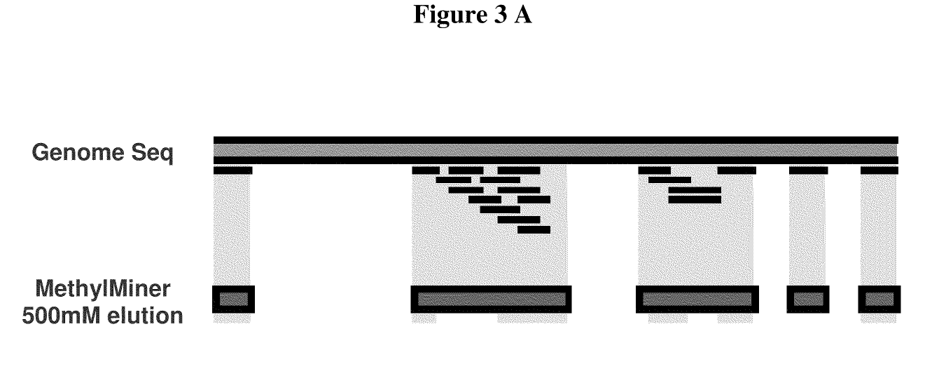

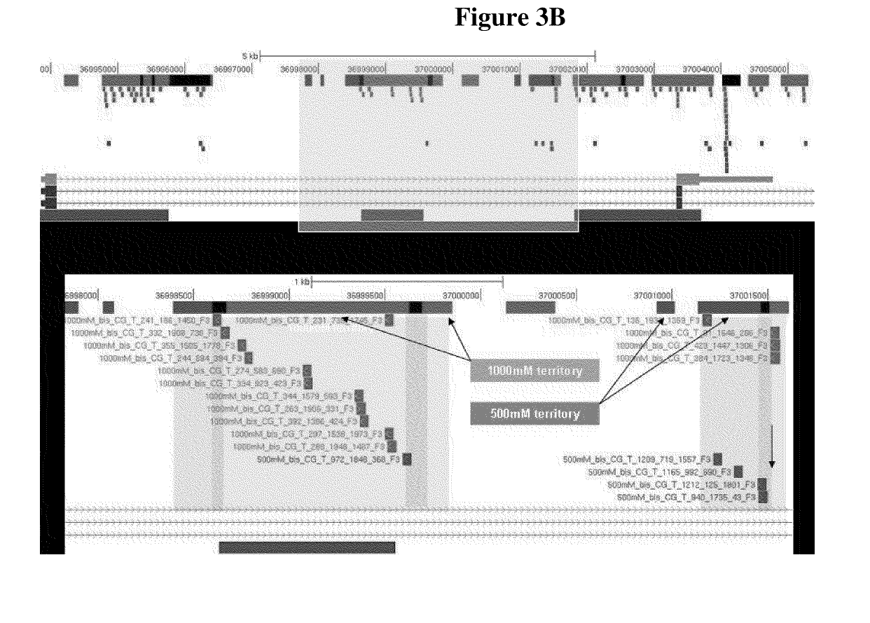

Nucleic acid molecules with various degrees of methylation may be separated from each other in the practice of the invention. As an example, FIG. 3B shows nucleic acid fragments which were eluted from MBD beads using 500 nM and 1,000 nM NaCl. Generally, when nucleic acid fragment size is relatively consistent (200 bps+/−30 bps), nucleic acid fragments with higher numbers of methylation sites will elute from solid matrices containing an MBP at higher NaCl concentrations. As a result, the use of elution solutions (e.g., buffers) containing differ NaCl concentrations (as well as other salts) may be employed to separate nucleic acid fragments based upon methylation density, in addition to the separation of methylated nucleic acid fragments from non-methylated nucleic acid fragments. Two applications of this principle are for (1) the separation of nucleic acid fragments by methylation density which differ in sequence and (2) the separation of nucleic acid fragments by methylation density which have the same of similar sequence. By similar is meant that the nucleic acid fragments contain at least a common subset of sequences. This is especially important when random fragmentation of large nucleic acid molecules is used to generate the nucleic acid fragments.

The separation of nucleic acid fragments which have the same of similar sequence by methylation density may be used to assess the average methylation density of a locus within a particular cell type. As an illustration, assume that a particular nucleic acid fragment is present in eluents containing 250 nM (low), 500 nM (medium), and 1,000 nM NaCl (high). Also assume that 30% of the nucleic acid fragments are located in the low salt eluent, 60% of the nucleic acid fragments are located in the low salt eluent, and 10% of the nucleic acid fragments are located in the low salt eluent. Thus, a ratio of 30:60:10 is shown from low, medium, and high salt eluents. Ratios of this type may be compared, for example, to the ratio found for a control cell or a cell which a particular phenotype (e.g., a tumor cell). Further, nucleic acid fragments present in each of the salt eluents may be subjected to bisulfite sequencing to determine methylation site locations and the methylation ratio at specific sites. For example, the C in the sequence ATACGAA may be methylated in 5% of the nucleic acid fragments in the low salt eluent, 25% of the nucleic acid fragments in the medium salt eluent, and 65% of the nucleic acid fragments in the high salt eluent; yielding a ratio of 5:25:65. Again, such ratios may be compared, for example, the ratio found for a control cell or a cell which a particular phenotype (e.g., a tumor cell). Thus, the invention includes methods for (1) identifying methylated regions of nucleic acid molecules (e.g., chromosomes), (2) determining the methylation density in specific regions of nucleic acid molecules, and (3) comparing the degree of methylation density in specific regions of nucleic acid molecules between different samples.

The invention also provides ratiometric data comparison methods. As one skilled in the art would understand and as implied by the above, the same sequence in each cell of a particular cell type may not always be methylated or unmethylated. Thus, the invention also includes methods by which the degree methylation of a particular sequence in cells in a sample may be compared. Such methods may be performed, for example, quantitatively or semi-quantitatively. An example of quantitative measurement would be the performance of bisulfite sequencing to determine the methylation ratio of a specific nucleotide sequence. An example of semi-quantitative measurement would be the determination of the prevalence/ratio of a particular nucleic acid fragment containing the specific nucleotide sequence in, for example, low, medium and high salt eluents, as, for example, described above. The invention may also be used to combine semi-quantitative and quantitative analysis. For example, semi-quantitative could be followed by quantitative analysis or semi-quantitative analysis could be followed by quantitative analysis when a particular result is obtained by semi-quantitative analysis. As an example, if semi-quantitative analysis yields a result which is consistent with that found in a negative control, it may be determined that quantitative analysis is not necessary.

Recovered DNA samples may be concentrated and cleaned up using ethanol precipitation. Precipitation is performed by adding 1 μl of glycogen (20 μg/μl), 1/10^{th} the sample volume of 3 M sodium acetate, pH 5.2, and 2 sample volumes of 100% ethanol. The sample is then mixed well and incubated for at least 2 hours at −80° C. Precipitated DNA is collected by centrifuging at 12,000×g for 15 minutes and discarding the supernatant. The pellet may then be washed by resuspending in 500 μl of 70% cold ethanol followed by centrifugation for 5 minutes at 12,000×g. The wash step should be repeated at least once. The pellet may then be partially air dried and then resuspended in an appropriate volume of buffer or water as needed for further processing.

It should be noted that about 10-fold enrichment has been observed on a mass-basis, i.e., about 1/10^{th} of a fragmented genomic sample can be recovered from a typical METHYLMINER™ based enrichment protocol. However, the sequence complexity, as determined by high throughput sequencing is typically reduced by 60-70%; this corresponds to 3- to 4-fold enrichment in terms of the unique sequences represented in the enriched material. Furthermore, since the affinity of MBD for methylated DNA can be modulated by ionic strength, fractionation of the captured DNA based on its degree of methylation may be performed with graded changes in ionic strength. DNA methylation in various genomic contexts, including regions of low, intermediate, or high CpG density influences gene regulation. Therefore, the ability to fractionate the genome according to the degree of methylation may be important for functional studies. This sub-fractionation may create an opportunity to generate higher degrees of enrichment for sub-populations of methylated sequences as well.

One approach to identify 5-methylcytidine is to use the bisulfite conversion reaction of cytosine to uracil described by Shapiro et al. (J. Amer. Chem. Soc. 92:422, 1970) and Hayatsu et al. (Biochemistry, 9:2858, 1970). 5-methylcytidine is resistant to this reaction so that when a polynucleotide treated with bisulfite is sequenced, non-methylated cytidine will be read as a U and 5-methylcytidine will be read as C. By comparing sequencing results of bisulfite treated and un-treated nucleotides, the location of 5-methylcytidine bases can be identified. This approach may be generally applicable to the analysis of any modified base where a differential sensitivity to a chemical modification can be demonstrated.

Bisulfite conversion protocols generally comprise four steps; denaturation, treatment with bisulfite to convert cytosine to uracil, desulfonation to remove sulfonic groups from converted uracils, and purification of the converted nucleic acid. Denaturation is a required step as it is known that double stranded DNA is resistant to bisulfite (Shapiro et al. J. Biol. Chem. 248:4060, 1973). Bisulfite initially reacts at the 6 position of cytosine to form cytosine sulfonate which then undergoes hydrolytic deamination to form uracil sulfonate. Treatment with alkali may then be used to remove the sulfonate group producing uracil.

Kits for the conversion of 5-methylcytidine to uridine are available commercially, for example the METHYLCODE™ Bisulfite Conversion Kit, (Life Technologies, Carlsbad, Calif.); EPITECT™ Bisulfite Kit, (Qiagen Inc., Valencia, Calif.); CPGENOME™ Fast DNA Modification Kit, (Millipore, Billerica, Mass.); and IMPRINTT™ DNA Modification Kit, (Sigma-Aldrich, St. Louis, Mo.).

The METHYLCODE™ Bisulfite Conversion Kit is used here as an illustrative example. From 500 μg to 2 μg of DNA may be processed using this protocol. The DNA sample is mixed with the sodium metabisulfite reagent and incubated at 98° C. for 10 minutes to denature the DNA followed by incubation at 64° C. for 2.5 hours for the bisulfite conversion to occur. The sample may then be stored at 4° C. for up to 20 hours prior to applying to a spin column and washing with binding buffer followed by treatment with desulphonation buffer for 15-20 minutes at room temperature. The spin column is washed twice with an ethanol containing wash buffer and the DNA eluted.

Other methods of modifying 5-methylcytidine may also be used. U.S. Patent Application No. 2006/0063189 describes sulfur nucleophiles which may be used as alternatives to bisulfite. The use of enzymatic methods to modify 5-methylcytidine are described in U.S. Patent Application Nos. 2006/0210990 and 2007/065824. The contents of these patent applications, as well as all other patent documents referred to herein, are incorporated herein in their entirety by reference. Other methods based on the conversion of C to U, by an alternative chemical or enzymatic agent will also be compatible with this workflow.

Once the methylated nucleic acid has been isolated and a portion converted by bisulfite or other treatment, both the converted and non-converted nucleic acid may be sequenced. There are currently four commercial systems available for ultra-high-throughput, massively parallel DNA sequencing: The SOLiD™ system (Applied BioSystems, Foster City, Calif.); the Genome Sequencer FLX system, commonly known as 454-sequencing (Roche Diagnostics, Indianapolis, Ind.); the Genome Analyzer (Illumina, San Diego, Calif.); and the Helicos Genetic Analysis System (Helicos Biosciences, Cambridge, Mass.).

Applied Biosystems' SOLiD approach for massively parallel DNA sequencing is based on sequential of cycles of DNA ligation (Shendure et al., Science 309: 1728-1732 (2005)). By this approach, immobilized DNA templates are clonally amplified on beads (emulsion PCR), which are plated at high density onto the surface of a glass flow cell. Sequence determination is accomplished by successive cycles of ligation of short defined labeled probes onto a series of primers hybridized to the immobilized template.

The 454-technology is based on conventional pyrosequencing chemistry carried out on clonally amplified DNA templates on microbeads individually loaded onto etched wells of a high-density optical plate (Margulies et al, Nature 437: 376-380. (2005)). Signals generated by each base extension are captured by dedicated optical fibers.

Illumina sequencing templates are immobilized onto a flow cell surface where they are clonally amplified in situ to form discrete sequence template clusters with densities up to ten-million clusters per square centimeter. Illumina-based sequencing is carried out using primer-mediated DNA synthesis in a step-wise manner in the presence of four proprietary modified nucleotides having a reversible 3′ di-deoxynucleotide moiety and a cleavable chromofluor. The 3′ di-deoxynucleotide moiety and the chromofluor are chemically removed before each extension cycle for successive base calling. Cycles of step-wise nucleotide additions from each template clusters are detected by laser excitation followed by imaging from which base calling is accomplished.

Helicos sequencing templates are immobilized on a proprietary surface without prior amplification to enable what is referred to as “True Single Molecule Sequencing”. This is achieved by polymerase-mediated sequence-specific incorporation of fluorescent nucleotide analogs that is observed by imaging laser-induced fluorescence (LIF). The imaging is done in cycles corresponding to a) the addition and enzymatic incorporation of one of the four base analogs, b) washing to remove free, non-incorporated bases, c) imaging to record LIF signal intensities and positions, and d) a cleavage step to eliminate the fluorescent signal. This process is repeated for each base analog and for each position along the template to create greater than 25-base reads.

Short sequencing reads may be mapped to a reference genome using conventional short read mapping software. Mapped reads may be analyzed for the distribution and depth of coverage over the reference genome. These statistics may be used to identify regions of the genome that have a depth of coverage equal to or in excess of the median read distribution, which corresponds to a territory map for a given experimental treatment. Different experiments may be used to produce individual territory maps of a reference genome for specific experimental conditions. Such maps can be combined to highlight similarities, differences and other combinations to produce a combined territory map for a series of experiments. These territory maps can be used to modify the reference genome base representation by maintaining the bases corresponding to the territory map regions and by converting bases outside of the territory map regions into non base characters. The territory map converted genome may then be used in further analysis. Exemplary territory maps are show in FIGS. 3A and 3B.

An exemplary workflow for analysis of data from METHYLMINER™ derived samples may include:

Mapping of unconverted and bisulfite-converted reads.

Mapping statistics and statistics on read coverage and depth.

Mapped reads output in BAM-format files.

Visualization of mapped reads on publicly available genome browsers.

Unconverted METHYLMINER™ reads may be mapped to a regular (unconverted) reference genome sequence. Bisulfite-converted reads may be mapped to a pair of appropriately converted reference sequences (forward and reverse conversions). For mapping bisulfite reads the following converted reference sequence pairs are recommended:

Pair 1:

Reference with all non-CpG C's converted to T's Reference with all non-CpG G's converted to A's

Or pair 2:

Reference with all C's converted to T's

Reference with all G's converted to A's

After the mapping steps are complete, the resulting BAM file with mapped reads can be visualized with compatible third-party commercial software tools and publicly-available genome browsers.

Besides viewing mapped reads in genome browsers, you may further analyze mapped reads with software available in the SOLiD™ development community or with other third-party tools.

Similarly, METHYLMINER™ bisulfite-converted mapped reads can be processed with peak-finding programs to identify regions of significant methylation. These reads can also be processed at nucleotide resolution to report the methylation status of individual C bases, for bases covered at sufficient read depth.

In summary, the invention involves the enrichment of methylated DNA sequences, followed by splitting the sample (or careful reproduction of the enriched sample), followed by analysis of the sample by high throughput sequencing with and without bisulfite conversion. The unconverted sample sequences provide a reduced complexity “map” or sub-genome of the “methylation territory” that the converted sequences can be aligned against. The combination of these datasets provides single-base resolution information on the pattern of cytidine methylation from the sample of interest at reduced cost, increased speed and high confidence.

The invention further provides methods for comparing samples. Sample comparison may be done in any number of ways or for any numbers of purposes (e.g., research, diagnostics, etc.). With respect to diagnostics, a sample (e.g., blood, biopsy tissue, etc.) may be obtained from a patient. Data may then be generated from the sample (e.g., a methylation territory map) and then compared to known samples. Known samples include control cells and cells which exhibit a particular phenotype (e.g., tumor cells).

The invention may be used for any number of applications. One set of exemplary applications is for the comparison of data derived from multiple sample sets. For purposes of illustration, tissue (e.g., muscle biopsy tissue) may be collected from three individual suspected of having a particular disease states (e.g., a sarcoma), then genomic DNA may be isolated, fragmented, size selected/purified; and then separated based upon methylation status. Once this has occurred, the relative amount of a particular sequence which is unmethylated and methylated may be determined. Further, the degree of methylation of the particular sequence may the be determined. The degree of methylation may then be compared to a negative control (e.g., normal muscle tissue) and a positive control (e.g., sarcoma tissue). The level of correlation between the samples and the controls may then be used to reach a determination of whether the sample tissue is more like the negative control or the positive control.

One area where the invention has applications is in the identification of imprinting disorders (e.g., disorders which result for the hypo- and/or hypermethylation of DNA). Examples of imprinting disorders include Angelman syndrome and Beckwith-Wiedemann syndrome which correlates with hypomethylation of PLAGL1 and GNAS loci (see, e.g., Tost, Methods Mol. Biol. 507:3-20 (2009)).

While the present teachings are described in conjunction with various embodiments, it is not intended that the present teachings be limited to such embodiments. On the contrary, the present teachings encompass various alternatives, modifications, and equivalents, as will be appreciated by those of skill in the art.

Further, in describing various embodiments, the specification may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process should not be limited to the performance of their steps in the order written, and one skilled in the art can readily appreciate that the sequences may be varied and still remain within the spirit and scope of the various embodiments.

The embodiments described herein, can be practiced with other computer system configurations including hand-held devices, microprocessor systems, microprocessor-based or programmable consumer electronics, minicomputers, mainframe computers and the like. The embodiments can also be practiced in distributing computing environments where tasks are performed by remote processing devices that are linked through a network.

It should also be understood that the embodiments described herein can employ various computer-implemented operations involving data stored in computer systems. These operations are those requiring physical manipulation of physical quantities. Usually, though not necessarily, these quantities take the form of electrical or magnetic signals capable of being stored, transferred, combined, compared, and otherwise manipulated. Further, the manipulations performed are often referred to in terms, such as producing, identifying, determining, or comparing.

Any of the operations that form part of the embodiments described herein are useful machine operations. The embodiments, described herein, also relate to a device or an apparatus for performing these operations. The systems and methods described herein can be specially constructed for the required purposes or it may be a general purpose computer selectively activated or configured by a computer program stored in the computer. In particular, various general purpose machines may be used with computer programs written in accordance with the teachings herein, or it may be more convenient to construct a more specialized apparatus to perform the required operations.

Certain embodiments can also be embodied as computer readable code on a computer readable medium. The computer readable medium is any data storage device that can store data, which can thereafter be read by a computer system. Examples of the computer readable medium include hard drives, network attached storage (NAS), read-only memory, random-access memory, CD-ROMs, CD-Rs, CD-RWs, magnetic tapes, and other optical and non-optical data storage devices. The computer readable medium can also be distributed over a network coupled computer systems so that the computer readable code is stored and executed in a distributed fashion.

The following examples are intended to illustrate but not limit the invention.

### EXAMPLES

### Example 1

The methylation pattern of a portion of human chromosome 21 was determined by analyzing a sample of human DNA (MCF-7 breast cancer cell line DNA from the BioChain Institute, Hayward, Calif.) As an initial step, a DNA sample enriched for methylated sequences was obtained by fractionating the sample using the MethylMiner™ methylated DNA enrichment kit (Invitrogen, Carlsbad, Calif.). The manufacturer's protocol was followed with the exception that the methylation enriched DNA was sequentially eluted from the beads in two fractions using 500 mM and 1000 mM NaCl solutions.

The enriched DNA sample was split into two portions and the first portion was submitted to sequencing using the SOLiD System (Applied Biosystems) with the SOLiD System Analysis Pipeline (“Corona Lite”) used for sequence analysis. Short reads were mapped to a reference genome using conventional short read mapping software. Mapped reads were analyzed for the distribution and depth of coverage over the reference genome. These statistics were used to identify regions of the genome that had a depth of coverage equal to or in excess of the median read distribution, which corresponded to a territory map for that experimental treatment. The territory map converted genome was then used for additional analysis.

The second portion of the enriched DNA sample was subjected to bisulfite conversion using the METHYLCODE™ Bisulfite Conversion Kit (Invitrogen, Carlsbad, Calif.) according to the manufacturer's instructions. The bisulfite converted DNA sample was then submitted to SOLiD sequencing. For bisulfite analyses, typically C residues in CpG doublets are protected by the addition of a methyl residue on the 5 carbon. All other C residues in the genome are not protected and are available for conversion to T residues through the bisulfite treatment methodology. To simplify the process of mapping, all C residues not present in a CpG doublet are converted to Ts in the territory map converted genome. This reduces the complexity of mapping bisulfite converted reads by reducing the number of errors required to align these reads with a fully converted reference genome in which every C is converted to T.

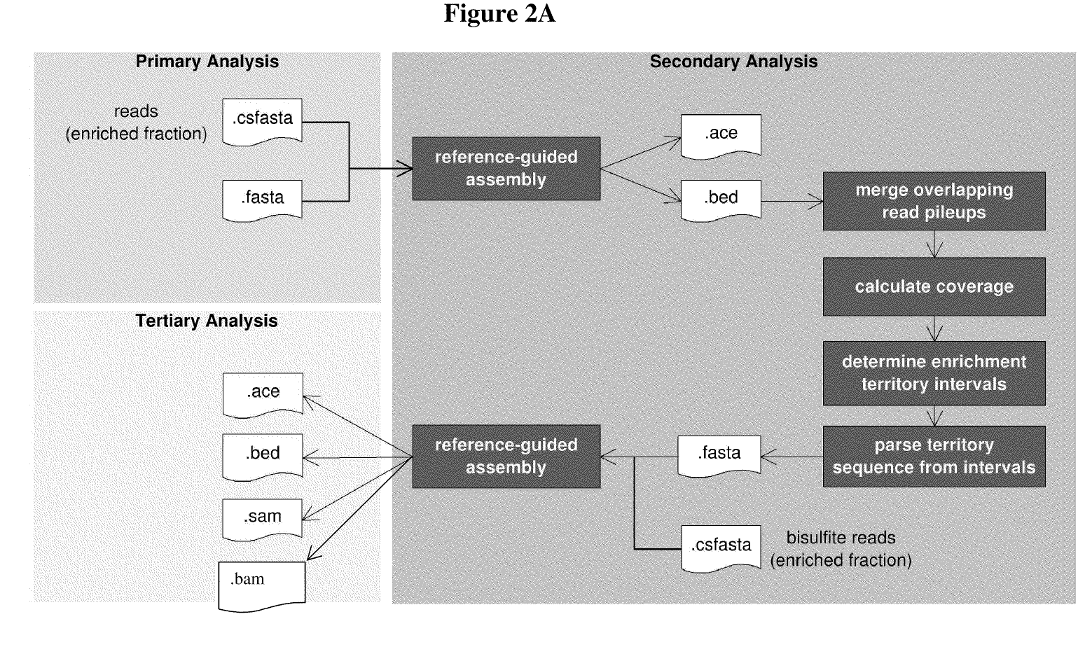

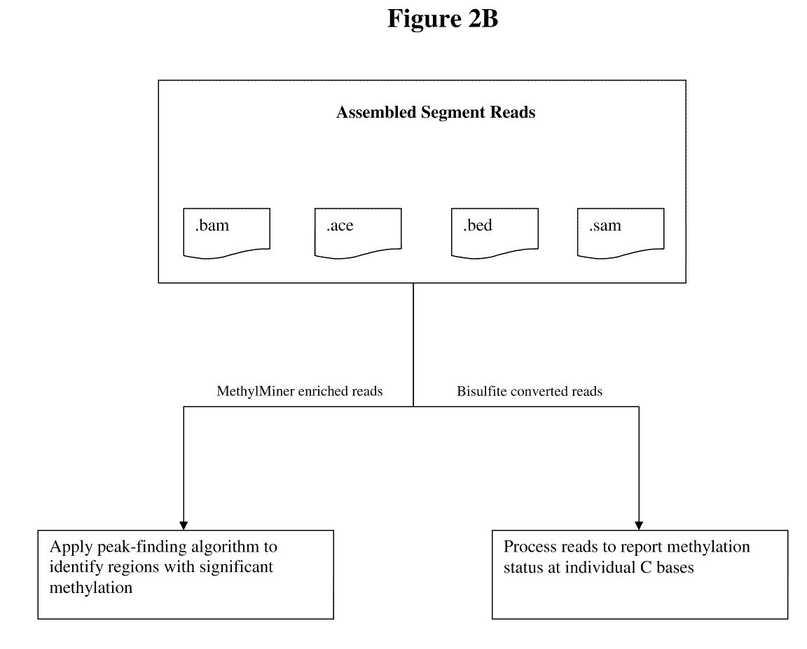

FIG. 2A depicts the computational steps used in the analysis of the sequencing reads. In reference to FIG. 2A, mapping enriched reads to a reference may comprise:

Align the enriched sequence reads to the reference genome using any reference-guided assembly software available.

Calculate the distribution and depth of coverage for the reads over the reference genome (i.e., read coverage).

Apply peak calling metrics to identify regions containing a read coverage equal to or in excess of the median read distribution (i.e., high coverage areas are identified).

Parse sequences from coverage intervals, these peaks become enriched methylation territory.

Parse gap sequences between enrichment intervals and mask gap sequences with X so that nothing can be aligned to these regions.

Stitch territory sequences together and masked sequences together to construct reference territory sequence for bisulfite read mapping. This becomes methylation territory reference for mapping.

Mapping bisulfite reads to territory may comprise:

Convert the reference sequence (enriched territory) to binary format.

Convert the bisulfite reads to binary format.

Align the bisulfite reads to the enrichment territory sequence (in color space); dump unaligned reads as FASTQ-formatted file.

Sort the aligned reads.

Create a multiple sequence alignment (reference-guided assembly) in ACE format.

Dump the multiple sequence alignment (reference-guided assembly) in FASTQ format.

Create a multiple sequence alignment (reference-guided assembly) in BED format.

Create a coverage plot.

A MethyMiner™ enriched methylation territory map and the use of this territory to align bisulfite converted SOLiD sequencing reads is depicted in FIG. 3. FIG. 3A illustrates a methylation territory derived from 500 mM MethyMiner™ eluted DNA sample (red bars) compared to a complete genomic reference sequence (green bar) and an illustration of bisulfite converted reads aligning to the territory (black bars). FIG. 3B shows Bisulfite-converted reads mapping within 500 mM and 1000 mM enriched fractions (i.e., methylated territories) respectively. Shown is a diagram of 500 mM (red bars) and 1000 mM (black bars) MethyMiner™ enriched methylated territories within a defined region of chromosome 21 and the bisulfite converted sequencing reads that map within each of these territories. Also shown are the areas where the 500 mM and 1000 mM territories overlap (black bars) and the bisulfite sequencing reads that map within this region. Green bars represent annotated CpG islands.

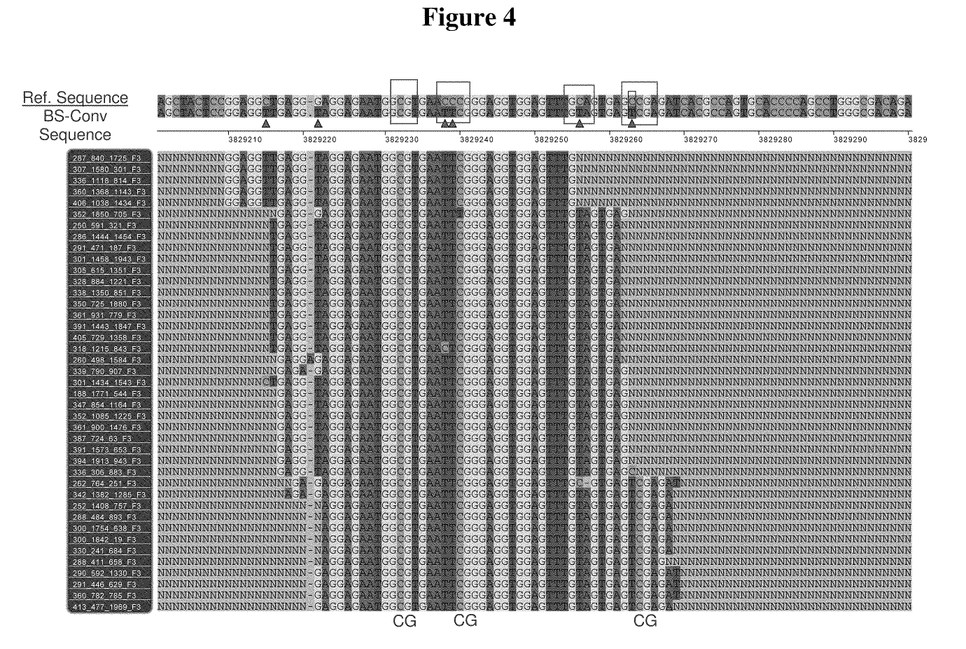

FIG. 4 shows a comparison of a reference sequence (top row) and a computationally determined bisulfite converted reference sequence (second row) for a portion of chromosome 21. Note that the Cs that were converted to Ts at positions 3829215, 3829222, 3829238, 3899239, 3829256 and 3829263 indicate the positions of non-methylated Cs and are all Cs that are not part of a CpG sequence. Below these two rows of reference sequence are 41 experimentally determined SOLiD reads of bisulfite converted DNA from the 500 mM NaCl elution described above. The experimentally determined reads have been aligned to the computationally determined bisulfite converted reference. This data indicates that the cytidine residues at positions 3829232, 3829240, and 3829264, each a member of a CG dinucleotide as indicated at the bottom of the figure, are all methylated in the original DNA sample since they persist as Cs in the majority of experimentally determined sequences that span this region.

Although the invention has been described with reference to the above example, it will be understood that modifications and variations are encompassed within the spirit and scope of the invention. Accordingly, the invention is limited only by the following claims.

What is claimed is:

1. A method of mapping methylated cytidine in a genome of an organism comprising,
(a) isolating methylated DNA fragments from the organism,
(b) sequencing a first portion of the methylated DNA fragments isolated from the genome of the organism thereby producing a first DNA sequence,
(c) sequencing a second portion of the methylated DNA isolated from the genome of the organism which has been treated such that non-methylated cytidine is converted to uridine thereby producing a second DNA sequence, and
(d) aligning the second DNA sequence with the first DNA sequence thereby producing a map of methylated cytidine in the genome of the organism.

2. The method of claim 1, wherein the methylated DNA fragments are isolated from the genome of the organism using a methyl binding protein.

3. The method of claim 1, wherein the methylated DNA fragments are isolated from the genome of the organism using antibodies specific for methylated DNA.

4. The method of claim 1, wherein non-methylated cytidine is converted to uridine by the use of bisulfite.

5. The method of claim 1, wherein the organism is a prokaryote.

6. The method of claim 1, wherein the organism is a eukaryote.

7. The method of claim 6, wherein the eukaryotic organism is a mammal.

8. The method of claim 7, wherein the mammalian eukaryotic organism is a human.

9. The method of claim 1, wherein the sequencing is performed by a high throughput method.

10. A method of mapping methylated cytidine in a genome of an organism comprising:
(a) isolating from the genome of the organism, methylated DNA fragments,
(b) splitting the isolated methylated DNA fragments into at least a first portion and a second portion,
(c) treating the first portion of isolated methylated DNA fragments such that non-methylated cytidine is converted to uridine,
(d) sequencing the first and second portions of isolated methylated DNA, and
(e) mapping the sequence of the first portion of the isolated methylated DNA to the sequence of the second portion of the isolated methylated DNA.

11. The method of claim 10, wherein the first and/or second portions of isolated methylated DNA are amplified prior to sequencing.

12. The method of claim 10, wherein the methylated DNA fragments are isolated from the genome of the organism using a methyl binding protein.

13. The method of claim 10, wherein the methylated DNA fragments are isolated from the genome of the organism using antibodies specific for methylated DNA.

14. The method of claim 10, wherein non-methylated cytidine is converted to uridine by the use of bisulfite.

15. The method of claim 10, wherein the organism is a prokaryote.

16. The method of claim 10, wherein the organism is a eukaryote.

17. The method of claim 16, wherein the eukaryotic organism is a mammal.

18. The method of claim 17, wherein the mammalian eukaryotic organism is a human.

19. The method of claim 10, wherein the sequencing is performed by a high throughput method.

20. A kit for mapping methylated cytidine in a genome of an organism comprising a methylated DNA binding substance bound to a solid support.

21. The kit of claim 20, further comprising one or more buffers for binding the methylated DNA to the DNA binding substance.

22. The kit of claim 21, further comprising one or more buffers for eluting the bound methylated DNA from the methylated DNA binding substance.

23. The kit of claim 22, further comprising reagents for converting methylated cytidine to uridine.

24. The kit of claim 23, further comprising a written manual describing data analysis procedures for mapping methylated cytidine in a genome of an organism.


SEQUENCE LISTING (1 sequence)

SEQ ID NO: 1 - DNA, 100 bp, Artificial
  Synthetic
agctactccggaggctgagggaggagaatggcgtgaacccgggaggtggagtttgcagtgagccgagatcacgccagtgcaccccagcctgggcgacaga